(19) United States
(12) Patent Application Publication
(10) Pub. No.: US 2025/0263796 A1
(43) Pub. Date: Aug. 21, 2025

(54) METHODS OF DETERMINING PTEN COPY NUMBER

(71) Applicant: ASTRAZENECA AB, Södertälje (SE)

(72) Inventors: Brian DOUGHERTY, Wilmington, DE (US); Paul LABROUSSE, Wilmington, DE (US); Daniel STETSON, Wilmington, DE (US)

(21) Appl. No.: 17/758,470

(22) PCT Filed: Jan. 6, 2021
(86) PCT No.: PCT/EP2021/050136

§ 371 Date: Jul. 7, 2022

Related U.S. Application Data
(60) Provisional application No. 62/957,913, filed on Jan. 7, 2020.

(51) Int. Cl. C12Q 1/6886

(52) U.S. Cl. CPC C12Q 1/6886; C12Q 2600/156

(57) ABSTRACT

Methods of determining PTEN copy number are described. The methods can include simultaneously amplifying three or more PTEN loci and two or more reference genes by digital PCR.

### CLAIM OF PRIORITY

This application claims priority to provisional U.S. 62/957,913, filed Jan. 7, 2020, the contents of which are hereby incorporated by reference in their entirety.

### BACKGROUND

PTEN (phosphatase and tensin homologue deleted on chromosome ten) is a tumor suppressor that is mutated or deleted in a wide variety of human cancers, including cancers of the breast, prostate, endometrium, ovary, brain, skin, thyroid, lung, bladder and colon, as well as melanoma, glioblastoma, and lymphoma. Indeed, PTEN is one of the most commonly mutated tumor suppressor genes in cancer. PTEN is a phosphatase that modifies proteins and lipids. Unlike most phosphatases, the main substrates of PTEN are inositol phospholipids generated by the activation of the phosphoinositide 3 kinase (PI3K). The inositol phospholipids generated by PI3K lead to downstream activation of Akt, mTOR and ultimately cell survival and protein translation. Therefore, PTEN is an important negative regulator of PI3K/Akt signaling and plays a critical role in tumor suppression. During tumor development, mutations and deletions of PTEN occur that inactivate its enzymatic activity leading to increased cell proliferation and reduced cell death. The prevalence of PTEN mutations in cancer underscores the significance of identifying instances of genetic changes to PTEN.

A decrease in the PTEN copy number can reflect a loss of PTEN expression and/or enzymatic activity. Most methods of determining PTEN copy number consider only one locus at a time, and therefore may not accurately reflect deletions at loci other than the one investigated. Accordingly, improved methods of determining PTEN copy number are desirable.

### SUMMARY

In one aspect, a method of determining PTEN copy number in a sample from a subject, includes: simultaneously amplifying three or more PTEN loci and two or more reference genes by digital PCR; calculating a ratio of PTEN amplification to reference gene amplification; and determining a PTEN copy number for the sample based on the ratio.

The ratio of PTEN amplification can be the ratio of: the concentration of all PTEN copies, divided by the number of PTEN loci amplified to: the concentration of all reference gene copies, divided by the number of reference genes amplified.

The ratio of PTEN amplification can be the ratio of: the concentration of copies of an individual PTEN locus to: the concentration of copies of an individual reference gene. In some embodiments, the method further includes determining the ratio of: the concentration of copies of each individual PTEN locus to: the concentration of copies of each individual reference gene.

The PTEN copy number can be the twice the ratio.

The three or more PTEN loci can include at least two of intron 2, intron 3, and intron 6. The three or more PTEN loci can include intron 2, intron 3, and intron 6, and optionally one or more additional PTEN loci.

In some embodiments, the method can further include determining the ratio of: the concentration of copies of a first reference gene to: the concentration of copies of a second reference gene.

In some embodiments, the method can further include diluting a portion of the sample with a corresponding sample of non-tumor origin.

The sample can include fresh frozen tumor tissue. The sample can includes cell free DNA. The sample can include formalin fixed parrafin embedded tumor or suspected tumor tissue.

The reference genes can include at least one of B3GNT2, ERCC3, OR5T1, RUFY2, and CC2D1B.

Other features, objects, and advantages will be apparent from the description and drawings, and from the claims.

### BRIEF DESCRIPTION OF THE DRAWINGS

FIG. 1A. Example of cluster formation at 1 ng input. Each circle at the bottom of graph represents droplets containing a unique reference gene locus, in this assay there are two targeted reference gene loci. Each of the three circles at the left of the graph represent droplets containing a PTEN locus. Droplets outside the circles contain targets from more than one locus.

FIG. 1B. Example of cluster formation at a higher input (approximately 5 ng).

### DETAILED DESCRIPTION

Described herein are methods of determining PTEN copy number by simultaneously measuring more than one, e.g., two or more PTEN loci in a sample. By measuring multiple PTEN loci simultaneously, the present methods correctly identify instances of PTEN deletion that could otherwise go unnoticed, and can more precisely quantify the extent of PTEN deletion. The methods are also amenable to a variety of sample sources, including fresh frozen tissue, cell free DNA, and formalin fixed parrafin embedded (FFPE) tissue samples.

Patient samples derived from plasma have very low circulating free DNA (cfDNA) yields. Often, the entire DNA sample must be added to the Next Generation Sequencing (NGS) library reaction. In addition, DNA derived from the tumor (ctDNA) are often present in smaller fractions relative to cfDNA normally found in plasma samples. Due to these confounding factors, the detection of gene deletions in plasma becomes very challenging. It is beneficial to have orthogonal confirmation with a technology such as ddPCR to ensure accuracy of the copy number results detected by NGS.

In embodiments, digital PCR (e.g., ddPCR) is used to simultaneously amplify more than one PTEN locus in a single experiment. The amplified material is then quantified, with the quantification being indicative of the copy number. Notably, the amplification and quantification occur locus-by-locus, so the copy number is measured independently at each locus. Thus, a change that affects only one locus can be distinguished from those that affect multiple loci.

In some embodiments, ddPCR is used to amplify and quantify PTEN loci. ddPCR allows simultaneous amplification of multiple loci with independent detection of each locus. Furthermore, one or more reference genes from the same sample is also simultaneously amplified and quantified with the PTEN loci. The reference genes are selected for having a known and stable copy number, providing a comparison for changes in PTEN copy number. In addition, discrepancies in the measured reference gene(s) may indicate a sample with high genomic instability.

In some embodiments, the quantification of amplified material is expressed as a concentration of copies of target DNA per unit volume. The measured concentrations of copies of PTEN loci and copies of reference genes can then be compared to arrive at a PTEN copy number (CN) in the sample.

In some embodiments, the total concentration of all PTEN loci is compared to the total concentration of the reference gene(s) to determine PTEN copy number. For example, when three PTEN loci and two reference genes are used,

$$PTEN CN = ( ( PTEN Conc ( copies / μL ) / 3 ) / ( Reference gene Conc ( copies ) / μL ) / 2 ) ) * 2$$

In some embodiments, the concentration of each PTEN locus is compared to the concentration of the reference gene(s) to determine PTEN copy number. In some embodiments, the concentration of each PTEN locus is compared to the concentration of each of the reference gene(s) to determine PTEN copy number. This technique provides independent measurements of copy number at each locus. For example, when three PTEN loci and two reference genes are used,

$$PTEN ( locus 1 ) CN = ( ( PTEN locus 1 Conc ( copies / μL ) ) / ( Reference gene 1 Conc ( copies / μL ) ) * 2$$

$$PTEN ( locus 1 ) CN = ( ( PTEN locus 1 Conc ( copies / μL ) ) / ( Reference gene 2 Conc ( copies / μL ) ) * 2$$

$$PTEN ( locus 2 ) CN = ( ( PTEN locus 2 Conc ( copies / μL ) ) / ( Reference gene 1 Conc ( copies / μL ) ) * 2$$

$$PTEN ( locus 2 ) CN = ( ( PTEN locus 2 Conc ( copies / μL ) ) / ( Reference gene 2 Conc ( copies / μL ) ) * 2$$

$$PTEN ( locus 3 ) CN = ( ( PTEN locus 3 Conc ( copies / μL ) ) / ( Reference gene 1 Conc ( copies / μL ) ) * 2$$

$$PTEN ( locus 3 ) CN = ( ( PTEN locus 3 Conc ( copies / μL ) ) / ( Reference gene 2 Conc ( copies / μL ) ) * 2$$

The concentration of copies of a first reference gene can be compared to the concentration of copies of one or more additional reference genes as an internal control, for example,

$$( ( Reference gene 1 Conc ( copies / μL ) ) / \\ ( Reference gene 2 Conc ( copies / μL ) ) * 2$$

Samples where there is 30% or greater variation in the reference genes may be indicative of genomic instability. In such instances, the experiment can be repeated using different reference genes.

In some embodiments, the PTEN loci can include intron 2, intron 3, and intron 6. The PTEN loci can be two or more of intron 2, intron 3, and intron 6. References genes can include B3GNT2, ERCC3, OR5T1, RUFY2, and CC2D1B.

In some embodiments, samples are of tumor origin or suspected tumor origin. Samples of tumor origin or suspected tumor origin can optionally be compared to matched samples of non-tumor origin.

In some embodiments where the sample is FFPE tissue, controls reflecting different levels of FFPE damage (mild, moderate, severe) can be used to compare the tested sample.

### EXAMPLES

The following examples are illustrative and not intended to be limiting. Other embodiments are within the scope of the following claims.

### Methods

### Reference Probe Selection

Due to the potential for genomic instability in samples derived from cancer patients, an analysis was performed to identify several stable genes across different cancer indications, to utilize as normal references. Genomic stability was described as percent diploid (showing two copies) in copy number estimates derived from NGS testing. The genes were ranked by the percent diploid score to provide the best candidates to use for a reference gene. Several genes were tested and found to be suitable candidates as shown in Table 1.

TABLE 1
List of reference genes for use in PTEN copy number assay

| Gene | Chromosome |
|---|---|
| ERCC3 | 2 |
| OR5T1 | 11 |
| RUFY2 | 10 |
| B3GNT2 | 2 |
| CC2D1B | 1 |

### PTEN Probe Selection

Selection of the PTEN loci were determined by analyzing the copy number data available in the cBio database. Briefly, data from patients with confirmed PTEN loss were analyzed for probe selection. Regions with a high prevalence of PTEN loss were selected. As no one region was found to be deleted across all the patient data, a combination of three different loci were selected to maximize the clinical sensitivity of the assay. By comparing chromosomal coordinates of the targeted PTEN loci to the chromosomal coordinates of the segment files from the dataset, it was found that the design would potentially detect losses in 99.3% (731/736) of the reported samples within the data sets.

### Sample Preparation

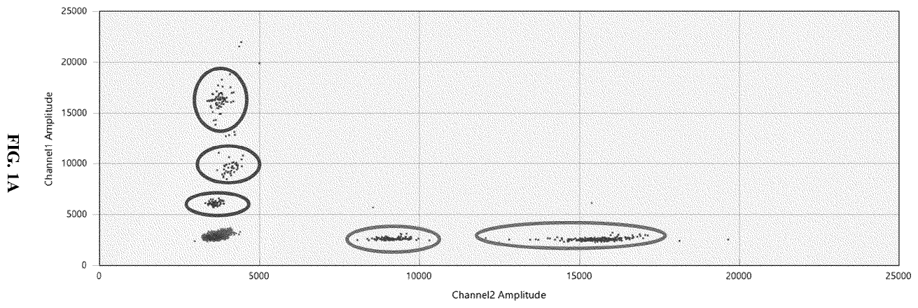

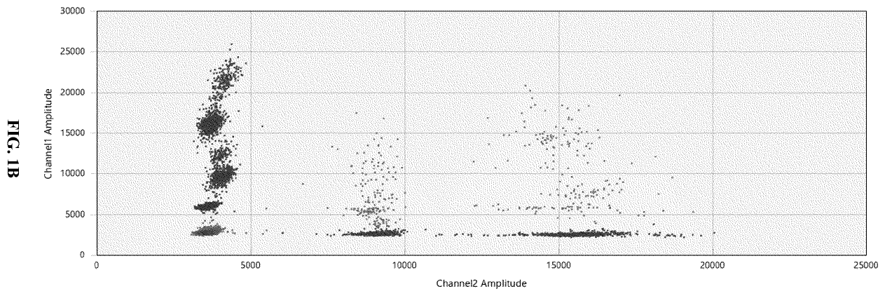

Dilute the test sample to 0.2 ng/μL, which results in a total of 1 ng input into the reaction. Using Poisson statistics, an input of 1 ng should give a distribution where the majority of the droplets contain one target. Manipulating the input concentration to where each droplet contains only one target would theoretically enhance cluster separation, which would make analysis more efficient (see FIGS. 1A and 1B).

FIG. 1A shows an example of cluster formation at 1 ng input, note that the number of dual occupied droplets is minimal. Each blue circle represents droplets containing a unique PTEN locus, in this assay there are three targeted PTEN loci. Each green circle represents droplets containing a unique reference gene locus, in this assay there are two targeted reference gene loci. Droplets outside the circles contain targets from more than one locus. FIG. 1B shows an example of cluster formation at a higher input (approximately 5 ng), note that the increased number of dual occupied droplets, which can confound cluster delineation.

### Preparation of the Master Mix

Mix the components listed in Table 2 together and mix well by vortexing. The initial concentration is 9 uM for both the forward and reverse primers, and 5 uM for each of the probes. The difference in the input volume for each of the PTEN loci (FAM channel) or the reference loci (HEX channel) is used to create differences in signal amplitude which creates separation between clusters. The total volume of the reaction is 22 μL, but only 20 μL is used for the droplet generation step (the remaining 2 μL is considered dead volume).

TABLE 2
Master mix components for the PTEN copy number assay

| Component | Vol. per rxn (μL) | [Final] |
|---|---|---|
| 2x ddPCR Supermix for probes | 11 | 1x |
| PTEN locus 1 Primer/probe (FAM) | 1.5 | 1.4x |
| PTEN locus 2 Primer/probe (FAM) | 1.1 | 1.0x |
| PTEN locus 3 Primer/probe (FAM) | 0.55 | 0.5x |
| reference primer/probe R1 (HEX) | 1.1 | 1.0x |
| reference primer/probe R2 (HEX) | 0.77 | 0.7x |
| Water | 0.98 | NA |
| Sample (0.2 ng/μL) | 5 | 1 ng |

### Droplet Generation

Droplet generation is performed using the BIO-RAD Automated Droplet Generator. The manufacturer's recommendations are followed during this step. The output of this step is a 40 μL volume containing approximately 20,000 droplets.

### Amplification

After the test sample has been partitioned into droplets, amplification of the reaction was carried out. Table 3 outlines the thermal cycling parameters used for the PTEN copy number assay.

TABLE 3
Thermal cycling parameters for the PTEN copy number assay

| Cycling step | Temp (° C.) | Time | Ramp rate | # cycles |
|---|---|---|---|---|
| Enzyme activation | 95 | 10 min | 2° C./sec | 1 |
| Denature | 94 | 30 sec | | 40 |
| Anneal/extend | 59 | 1 min | | |
| Enzyme Deactivation | 98 | 10 min | | |
| Hold | 4 | infinite | 1° C./sec | 1 |

### Droplet Reading

The individual droplets were interrogated using the BIO-RAD QX200 Droplet Reader. The data from these reads were used in result calling.

### Example 1

A tumor sample (FFPE) was assayed by ddPCR at three PTEN loci and two reference genes (B3GNT2 and ERCC3). The sample assayed at 100% concentration and repeated at dilutions with non-tumor material. Initial tumor cellularity was 70%. Table 4 summarizes the results:

TABLE 4
illustrative CN determination

| Dilution | Est. | CN using B3GNT2 | | | CN using ERCC3 | | | Aggregate |
|---|---|---|---|---|---|---|---|---|
| factor | tumor % | Intron 2 | Intron 6 | Intron 3 | Intron 2 | Intron 6 | Intron 3 | CN est. |
| — | 0.0 | 2.24 | 1.60 | 1.92 | 2.01 | 1.43 | 1.71 | 1.85 |
| 1 | 0.7 | 2.28 | 1.67 | 2.04 | 1.92 | 1.41 | 1.72 | 1.81 |
| 2.5 | 1.8 | 2.16 | 1.62 | 1.91 | 1.99 | 1.49 | 1.76 | 1.81 |
| 5 | 3.5 | 2.30 | 1.56 | 2.08 | 2.18 | 1.48 | 1.97 | 1.93 |
| 10 | 7.0 | 2.42 | 1.61 | 2.01 | 2.18 | 1.46 | 1.82 | 1.89 |
| 20 | 14.0 | 2.33 | 1.46* | 1.96 | 2.14 | 1.34* | 1.80 | 1.81 |
| 30 | 21.0 | 2.23 | 1.36* | 1.93 | 2.07 | 1.26* | 1.79 | 1.76** |
| 50 | 35.0 | 1.91* | 1.09* | 1.74* | 1.76* | 1.00* | 1.60* | 1.51* |
| 100 | 70.0 | 1.76* | 0.63* | 1.57* | 1.51* | 0.54* | 1.35* | 1.23* |

*predicted PTEN loss
**indeterminate

### Example 2

Tissue and plasma samples from ER⁺ metastatic breast cancer patients were collected during a phase I trial of fulvestrant and capivasertib. For some patients, two samples were collected at different timepoints (designated ‘baseline’ and ‘later’ in the Table below).

The tissue samples were analyzed for PTEN status by Next Generation Sequencing (NGS), at the time of collection. The plasma (ctDNA) samples were analysed for PTEN status by low pass whole genome sequencing (LP-WGS) analysis performed with iCHOR CNA; and separately by ddPCR as described herein. Results are summarized in Table 5.

TABLE 5

| | | PTEN | | PTEN | PTEN |
|---|---|---|---|---|---|
| | Sample | status | Tumor | status | status |
| Patient | time | (tissue | fraction | (ctDNA, iCHOR | (ctDNA, |
| ID | point | NGS) | (%) | CNA LP-WGS) | ddPCR) |
| 951 | Baseline | PTENdel | 0 | PTENdel | PTENdel |
| | Later | PTENdel | 19 | PTENdel | PTENdel |
| 964 | Baseline | PTENdel | 19 | PTENdel | PTENdel |
| | Later | PTENdel | 31 | PTENdel | PTENdel |
| 991 | Baseline* | PTENdel | 17 | PTENdel | PTENdel |
| | Later* | PTENdel | 15 | PTEN copy | PTENdel |
| | | | | neutral | |
| 941 | Later | PTENdel | 11 | PTEN copy | PTENdel |
| | | | | neutral | |
| 879 | Later | PTENdel | 22 | PTEN copy | PTENdel |
| | | | | neutral | |
| 963 | Later | PTENdel | 19 | PTEN copy | PTENdel |
| | | | | neutral | |
| 962 | Baseline | PTENdel | 11 | PTEN copy | PTENdel |
| | | | | neutral | |

*likely non-shedder

Table 5 shows that the determination of PTEN status by ctDNA LP-WGS miscategorized several patients as PTEN-copy neutral, disagreeing with the result obtained by NGS from tissue samples. In contrast, the ddPCR method uniformly agreed with the tissue sample results. The Tumor Fraction estimate was derived from the iCHOR CNA analysis of the LP-WGS data.

1. A method of determining PTEN copy number in a sample from a subject, comprising:
simultaneously amplifying three or more PTEN loci and two or more reference genes by digital PCR;
calculating a ratio of PTEN amplification to reference gene amplification; and
determining a PTEN copy number for the sample based on the ratio.

2. The method of claim 1, wherein the ratio of PTEN amplification is the ratio of:
the concentration of all PTEN copies, divided by the number of PTEN loci amplified
to:
the concentration of all reference gene copies, divided by the number of reference genes amplified.

3. The method of claim 1, wherein the ratio of PTEN amplification is the ratio of:
the concentration of copies of an individual PTEN locus
to:
the concentration of copies of an individual reference gene.

4. The method of claim 3, further comprising determining the ratio of:
the concentration of copies of each individual PTEN locus
to:
the concentration of copies of each individual reference gene.

5. The method of claim 1, wherein the PTEN copy number is the twice the ratio.

6. The method of claim 1, wherein the three or more PTEN loci include at least two of intron 2, intron 3, and intron 6.

7. The method of claim 6, wherein the three or more PTEN loci include intron 2, intron 3, and intron 6, and optionally one or more additional PTEN loci.

8. The method of claim 1, further comprising determining the ratio of:
the concentration of copies of a first reference gene
to:
the concentration of copies of a second reference gene.

9. The method of claim 1, further comprising diluting a portion of the sample with a corresponding sample of non-tumor origin.

10. The method of claim 1, wherein the sample includes fresh frozen tumor tissue.

11. The method of claim 1, wherein the sample includes cell free DNA.

12. The method of claim 1, wherein the sample includes formalin fixed parrafin embedded tumor or suspected tumor tissue.

13. The method of claim 1, wherein the reference genes include at least one of B3GNT2, ERCC3, OR5T1, RUFY2, and CC2D1B.
